(19) United States
(12) Patent Application Publication
(10) Pub. No.: US 2005/0002876 A1
(43) Pub. Date: Jan. 6, 2005

(54) Oral care methods and products

(76) Inventors: Richard L. Yukl, Denver, CO (US); David Bar-Or, Englewood, CO (US)

Correspondence Address: SHERIDAN ROSS PC, 1560 BROADWAY, SUITE 1200, DENVER, CO, 80202, US

(21) Appl. No.: 10/840,943

(22) Filed: May 7, 2004

Related U.S. Application Data
(60) Provisional application No. 60/468,797, filed on May 7, 2003.

(51) Int. Cl. A61K 7/22; A61K 31/13

(52) U.S. Cl. 424/54; 514/674

(57) ABSTRACT

The invention provides methods, oral care products and kits for treating mouth tissues of an animal. In particular, the invention provides methods, oral care products and kits which use or comprise a non-peptide polyamine chelating agent, most preferably trientine, or a physiologically-acceptable salt thereof, which can inhibit the release of pro-inflammatory cytokines, particularly interleukin 8, from cells located in tissues of the mouth and can reduce the damage done by reactive oxygen species (ROS) to such tissues.

### CROSS-REFERENCE TO RELATED APPLICATIONS

This application claims benefit of provisional application No. 60/468,797, filed May 7, 2003, the complete disclosure of which is incorporated herein by reference.

### FIELD OF THE INVENTION

The invention provides methods, oral care products and kits for treating mouth tissues of an animal. In particular, the invention provides methods, oral care products and kits which use or comprise a non-peptide polyamine chelating agent, most preferably trientine, or a physiologically-acceptable salt thereof, which can inhibit the release of pro-inflammatory cytokines, particularly interleukin 8, from cells located in tissues of the mouth and can reduce the damage done by reactive oxygen species (ROS) to such tissues.

### BACKGROUND

Reactive oxygen species (ROS) include free radicals (e.g., superoxide anion and hydroxyl, peroxyl, and alkoxyl radicals) and non-radical species (e.g., singlet oxygen and hydrogen peroxide). ROS are capable of causing extensive molecular, cellular and tissue damage, and they have been reported to play a major role in a variety of diseases and conditions. Indeed, ROS have been implicated in over 100 diseases and pathogenic conditions, and it has been speculated that ROS may constitute a common pathogenic mechanism involved in all human diseases. Stohs, J. Basic Clin. Physiol. Pharmacol., 6, 205-228 (1995). For reviews describing ROS, their formation, the mechanisms by which they cause molecular, cellular and tissue damage, and their involvement in numerous diseases and disorders, see, e.g., Manso, Rev. Port. Cardiol., 11, 997-999 (1992); Florence, Aust. N Z J. Opthalmol., 23, 3-7 (1992); Stohs, J. Basic Clin. Physiol. Pharmacol., 6, 205-228 (1995); Knight, Ann. Clin. Lab. Sci., 25, 111-121 (1995); Kerr et al., Heart & Lung, 25, 200-209 (1996); Roth, Acta Chir. Hung., 36, 302-305 (1997).

Metal ions, primarily transition metal ions, can cause the production and accumulation of ROS. In particular, copper and iron ions released from storage sites are one of the main causes of the production of ROS following injury, including ischemia/reperfusion injury and injury due to heat, cold, trauma, excess exercise, toxins, radiation, and infection. Roth, Acta Chir. Hung., 36, 302-305 (1997). Copper and iron ions, as well as other transition metal ions (e.g., vanadium, and chromium ions), have been reported to catalyze the production of ROS. See, e.g., Stohs, J. Basic Clin. Physiol. Pharmacol., 6, 205-228 (1995); Halliwell et al., Free Radicals In Biology And Medicine, pages 1-19 (Oxford university 1989); Marx et al., Biochem. J., 236, 397-400(1985); Quinlan et al., J. Pharmaceutical Sci., 81, 611-614 (1992). Other transition metal ions (e.g., cadmium, mercury, and nickel ions) and other metal ions (e.g., arsenic and lead ions) have been reported to deplete some of the molecules of the natural antioxidant defense system, thereby causing an increased accumulation of ROS. See, e.g., Stohs, J. Basic Clin. Physiol. Pharmacol., 6, 205-228 (1995).

ROS may be present in the mouth for a variety of reasons. For instance, ROS are present in the mouth as a result of the use of tobacco products, exposure to environmental agents, exposure to radiation, and the use of oral care products comprising tooth whitening agents that liberate active oxygen or hydrogen peroxide. See, e.g., U.S. Pat. Nos. 5,906,811, 6,228,347, and 6,270,781. ROS may also be present in the mouth as a result of diseases and conditions that involve inflammation and/or infection, including gingivitis, periodontitis, injuries, surgeries, tooth extractions, cold sores, canker sores and ulcers. See, e.g., U.S. Pat. Nos. 6,228,347 and 6,270,781. Finally, although the normal pH of saliva is 7.2, acidic conditions often are present in the mouth, e.g., as a result of the breakdown of foods, especially carbohydrates. See, e.g., U.S. Pat. No. 6,177,097. Acidic conditions promote the release of copper ions from proteins to which they are bound and, as discussed above, free copper ions can cause the production of ROS. The ROS present in the mouth can cause damage to the tissues of the mouth. For instance, in inflammatory periodontal diseases, ROS and elevated levels of free iron and copper ions have been found in periodontal pockets, suggesting a significant role for ROS in periodontal tissue destruction. See, e.g., Waddington et al., Oral Dis., 6: 138-151 (2000).

Interleukin 8 (IL-8) is a pro-inflammatory cytokine and a potent chemoattractant and activator of neutrophils. It has also been reported to be a chemoattractant and activator of T-lymphocytes and eosinophils. IL-8 is produced by immune cells (including lymphocytes, neutrophils, monocytes and macrophages), fibroblasts and epithelial cells. Reports indicate an important role for IL-8 in the pathogenesis of many inflammatory disorders, including gingivitis and periodontal disease. Sfakianakis et al., J. Periodontal Res., 37(2):154-160 (April 2002), Fitzgerald et al., Oral Microbiol. Immunol., 10(5):297-303 (October 1995), and Takigawa et al., J. Periodontol., 65(11):1002-1007 (November 1994). It has recently been found that endothelial cells secrete markedly elevated levels of IL-8 after exposure to a physiologically relevant concentration of copper (see co-pending U.S. application Ser. No. 10/186,168, filed Jun. 27, 2002, now published U.S. application No. US20030130185, published Jul. 10, 2003, PCT WO 03/043518, published May 30, 2003, and Bar-Or, Thomas, Yukl, Rael, Shimonkevitz, Curtis and Winkler, “Copper Stimulates the Synthesis and Release of Interleukin-8 in Human Endothelial Cells: A Possible Early Role in Systemic Inflammatory Responses,” Shock, 20(2):154-158 (August 2003)). Also, copper catalyzes the production ofROS which cause the release of IL-8 from other cell types. See, e.g., Kennedy et al., Am. J. Respir. Cell. Mol. Biol., 19(3):366-378 (1998).

### SUMMARY OF THE INVENTION

The invention provides a method of inhibiting the release of pro-inflammatory cytokines from cells located in a tissue of an animal's mouth. The method comprises contacting the tissue with an effective amount of a non-peptide polyamine chelating agent or a physiologically-acceptable salt thereof.

The invention also provides a method of treating inflammation of a tissue of an animal's mouth. This method comprises contacting the tissue with an effective amount of a non-peptide polyamine chelating agent or a physiologically-acceptable salt thereof.

The invention further provides a method of treating an inflammatory disease or condition of a tissue of an animal's mouth. The method comprises contacting the tissue with an effective amount of a non-peptide polyamine chelating agent or a physiologically-acceptable salt thereof.

The invention also provides a method of reducing the damage done by reactive oxygen species to a tissue of an animal's mouth. The method comprises contacting the tissue with an effective amount of a non-peptide polyamine chelating agent or a physiologically-acceptable salt thereof.

The invention further provides a method of whitening one or more teeth of an animal. The method comprises contacting the one or more teeth, another tissue of the animal's mouth, or both with an effective amount of a non-peptide polyamine chelating agent or a physiologically-acceptable salt thereof.

The invention also provides a method of treating a tissue of an animal's mouth. The method comprises contacting the tissue with from about 0.002 mg to about 2.20 mg of trientine or a physiologically-acceptable salt thereof.

The invention further provides amethod of treating a disease or condition of a tissue of an animal's mouth. The method comprises contacting the tissue with from about 0.002 mg to about 2.20 mg of trientine or a physiologically-acceptable salt thereof.

The invention also provides an oral care device. The device comprises a non-peptide polyamine chelating agent or a physiologically-acceptable salt thereof.

The invention further provides an oral care composition. The composition comprises a pharmaceutically-acceptable carrier and from about 0.001% to about 25% by weight of a non-peptide polyamine chelating agent or a physiologically-acceptable salt thereof.

The invention also provides a tooth whitening composition. The composition comprises a pharmaceutically-acceptable carrier and a non-peptide polyamine chelating agent or a physiologically-acceptable salt thereof.

The invention further provides a kit. The kit comprises an oral care product, the oral care product comprising a non-peptide polyamine chelating agent or a physiologically-acceptable salt thereof. The oral care product may be an oral care device or an oral care composition.

### BRIEF DESCRIPTION OF THE DRAWINGS

FIG. 1: Graph showing the effects of Syprine® trientine hydrochloride on copper-induced release of IL-8 from human umbilical vein endothelial cells (HUVEC).

### DETAILED DESCRIPTION OF THE PRESENTLY-PREFERRED EMBODIMENTS

As used herein, “non-peptide, polyamine chelating agent” means a compound that: (i) is not a peptide or protein; (ii) contains three or more, preferably four or more, nitrogen atoms (N's), with the N's being separated by one or more, preferably two or three, carbon atoms (C's); and (iii) chelates copper ions, iron ions or both, and may chelate other metal ions, including particularly transition metal ions. The N's and C's may be unsubstituted, or one or more of the N's and/or C's may be substituted with substituents that do not interfere with chelation of metal ions and that do not, in combination, produce a chemically unstable configuration. Many linear and cyclic non-peptide polyamine chelating agents are known, and they are available commercially or can be made by known methods. See, e.g., U.S. Pat. Nos. 5,101,041, 5,422,096, 5,906,996 and 6,264,966 and PCT applications WO 00/21941 and WO 99/39706, the complete disclosures of which are incorporated herein by reference.

Preferred nonpeptide, polyamine chelating agents for use in the practice of the present invention are those having the following formula:
NR¹R²[—(CR³R⁴)ₓ—NR¹]_{y}—(CR³R⁴)ₓ—NR¹R².

Each R¹, R², R³ and R⁴ may be the same or different, and each R¹, R², R³ and R⁴ may be H, alkyl (straight-chain or branched), alkenyl, aryl, arylalkyl, alkoxy, alkenoxy, aryloxy, alkyl interrupted by one or more oxygen atoms (—O—), alkyl interrupted by one or more nitrogen atoms (—N—), alkenyl interrupted by one or more oxygen atoms (—O—), alkenyl interrupted by one or more nitrogen atoms (—N—), aryloxyalkyl, alkoxyaryl, aminoalkyl, aminoalkenyl, aminoaryl, aminoarylalkyl, hydroxyalkyl, hydroxyalkenyl, hydroxyaryl or hydroxyarylalkyl. In addition, each R¹ and each R² may be:
—CR⁵R⁶—(CR⁷R⁸)_{z}—R⁹.

Each R⁵, R⁶ and R⁷ may be the same or different, and each R⁵, R⁶ or R⁷ may be H, alkyl (straight-chain or branched), alkenyl, aryl, arylalkyl, alkoxy, alkenoxy, aryloxy, alkyl interrupted by one or more oxygen atoms (—O—), alkyl interrupted by one or more nitrogen atoms (—N—), alkenyl interrupted by one or more oxygen atoms (—O—), alkenyl interrupted by one or more nitrogen atoms (—N—), aryloxyalkyl, alkoxyaryl, aminoalkyl, aminoalkenyl, aminoaryl, aminoarylalkyl, hydroxyalkyl, hydroxyalkenyl, hydroxyaryl or hydroxyarylalkyl.

R⁸ may be H, hydroxy, amino, alkyl (straight-chain or branched), alkyl interrupted by one or more oxygen atoms (—O—), alkoxy, aryl, aryloxyalkyl or alkoxyaryl.

R⁹ may be any of the following: —COOR¹⁰, —CON(R¹⁰)₂, —OR¹⁰, —C(OR¹⁰)₃, —COR¹⁰, —N(R¹⁰)₂, alkyaryl, or alkylheteroaryl.

R¹⁰ may be H, alkyl (straight-chain or branched), aryl or heteroaryl.

Each x may independently be 2 or 3, y is 1-10 (preferably 1-5, more preferably 2-4, most preferably 2), and z is 0-6.

The physiologically-acceptable salts of the non-peptide polyamine chelating agents can also be used in the practice of the invention. Physiologically-acceptable salts include conventional non-toxic salts, such as salts derived from inorganic acids (such as hydrochloric, hydrobromic, sulfuric, phosphoric, nitric, and the like), organic acids (such as acetic, propionic, succinic, glycolic, stearic, lactic, malic, tartaric, citric, glutamic, benzoic, salicylic, and the like), or inorganic and organic bases (such as the hydroxide, carbonate or bicarbonate of a pharmaceutically-acceptable metal cation). The salts are prepared in a conventional manner, e.g., by neutralizing the free base form of the compound with an acid.

The binding of metal ions, particularly copper and/or iron ions, by the non-peptide polyamine chelating agents and their physiologically-acceptable salts inhibits (i.e., reduces or prevents) the production of ROS and/or the accumulation of ROS caused by these metal ions. As a result, the damage that can be caused by ROS in the absence of the binding of the metal ions is reduced. In addition, the binding of copper ions by the non-peptide polyamine chelating agents and their physiologically-acceptable salts inhibits (reduces or prevents) the release of pro-inflammatory cytokines, particularly IL-8, from cells. Thus, the non-peptide polyamine chelating agents and their physiologically-acceptable salts can be used to treat inflammation and inflammatory disease and conditions of a tissue of an animal's mouth.

Preferred non-peptide polyamine chelating agents are those wherein R¹, R², R³ and R⁴ are all H, each x is independently 2 or 3, and y is 1-5. Such preferred non-peptide polyamine chelating agents include diethylenetriamine, trientine, tetraethylenepentamine, pentaethylenehexamine, hexaethyleneseptamine, dipropylenetriamine, tripropylenetetramine, tetrapropylenepentamine, pentapropylenehexamine, hexapropyleneseptamine, N,N′-bis(2-aminoethyl)propanediamine and N,N′-bis(2-aminopropyl)ethanediamine.

Highly preferred for use in the practice of the present invention is trientine. Trientine is also known as triethylenetetramine, N,N′-bis(2-aminoethyl)-1,2-ethanediamine and other names. See The Merck Index, page 1382, entry 9483 (10^{th} ed. 1983). It has the chemical formula NH₂—CH₂—CH₂—NH—CH₂—CH₂—NH—CH₂—CH₂—NH₂. Trientine and its physiologically-acceptable salts are available commercially from, e.g., Merck & Co., Inc., USA, Research Diagnostics Inc., USA, Akzo Nobel Functional Chemicals, Sweden, Ciba Specialty Chemicals, USA, Diamines & Chemicals Ltd., India, Fluka Chemie AG, Switzerland, Guangzhou Chemical Reagent Factory, China, Huntsman Chemical Corp., USA, ITI International Trade Inc., USA, Seratec S.A.R.L., France, Dow Chemical Co., USA, Tosoh Corporation, Japan, and Union Carbide Corp., USA. Trientine can also be prepared as described in U.S. Pat. Nos. 5,225,599, 4,980,507, 4,827,037, 4,720,588, 4,404,405 and 4,323,558 and the references cited in The Merck Index, page 1382, entry 9483 (10^{th} ed. 1983). The physiologically-acceptable salts of trientine can be prepared in a conventional manner, e.g., by neutralizing the free base form of the compound with an acid.

Trientine and its physiologically-acceptable salts are copper chelators. They bind Cu(II) with high-affinity and also bind Cu(I). In addition, they bind Ni(II), Zn(II), Co(II), and Fe(III). The binding of copper and iron ions by trientine and its physiologically-acceptable salts inhibits (i.e., reduces or prevents) the production of ROS and/or the accumulation of ROS caused by these metal ions. As a result, the damage that can be caused by ROS in the absence of the binding of the copper and iron ions by trientine is reduced. In addition, the binding of copper ions by trientine inhibits (reduces or prevents) the release of pro-inflammatory cytokines, particularly IL-8, from cells. Thus, trientine can be used to treat inflammation and inflammatory disease and conditions of a tissue of an animal's mouth.

In particular, it has been found that trientine is highly effective in treating gingivitis and periodontitis. Comparisons of data generated using trientine with published reports of other treatments for gingivitis and periodontitis, including antimicrobial compounds (chlorhexidine) and collegenase inhibitors (low dose doxycyline hyclate), suggest that trientine is several times more effective than these other treatments and produces its effects over a shorter treatment period and with fewer side effects. Moreover, the effective treatment of gingivitis and periodontitis with trientine was achieved using a very low dose of the compound. In addition and quite surprisingly, preliminary data indicate that trientine may be effective in whitening heavily stained teeth in the absence of added whitening agents.

The invention also provides oral care products comprising a non-peptide polyamine chelating agent or a physiologically-acceptable salt thereof. Oral care products include oral care compositions and oral care devices.

Oral care compositions of the invention include washes, rinses, gargles, solutions, drops, emulsions, suspensions, liquids, pastes, gels, ointments, creams, sprays, powders, tablets, gums, lozenges, mints, films, patches, and tooth whitening compositions. Oral care compositions of the invention include compositions intended for use by consumers and patients and compositions intended for use by dental professionals (e.g., dental hygienists, dentists and oral surgeons).

The oral care compositions of the invention will comprise a non-peptide polyamine chelating agent or a physiologically-acceptable salt thereof as active ingredient in admixture with one or more pharmaceutically-acceptable carriers. Oral care compositions of the invention will generally comprise from about 0.001% to about 25%, preferably from about 2.5% to about 12.5%, most preferably from about 5.0% to about 6.0%, by weight of a non-peptide polyamine chelating agent or a physiologically-acceptable salt thereof. The oral care compositions of the invention may also comprise one or more other acceptable ingredients, including other active compounds and/or other ingredients conventionally used in oral care compositions. Each carrier and ingredient must be “acceptable” in the sense of being compatible with the other ingredients of the formulation and not injurious to the animal.

Suitable ingredients, including pharmaceutically-acceptable carriers, for use in oral care compositions, and methods of making and using oral care compositions, are well known in the art. See, e.g., U.S. Pat. Nos. 4,847,283, 5,032,384, 5,043,183, 5,180,578, 5,198,220, 5,242,910, 5,286,479, 5,298,237, 5,328,682, 5,407,664, 5,466,437, 5,707,610, 5,709,873, 5,738,840, 5,817,295, 5,858,408, 5,876,701, 5,906,811, 5,932,193, 5,932,191, 5,951,966, 5,976,507, 6,045,780, 6,197,331, 6,228,347, 6,251,372, and 6,350,438, PCT applications WO 95/32707, WO 96/08232 and WO 02/13775, and EP applications 471,396, the complete disclosure of all of which are incorporated herein by reference. Conventional ingredients used in oral care compositions include water, alcohols, humectants, surfactants, thickening agents, abrasives, flavoring agents, sweetening agents, antimicrobial agents, anti-caries agents, anti-plaque agents, anti-calculus agents, pH-adjusting agents, and many others.

The water used in oral care compositions should preferably be of low ion content. It should also be free of organic impurities.

The alcohol must be nontoxic. Preferably the alcohol is ethanol. Ethanol is a solvent and also acts as an antibacterial agent and as an astringent.

Humectants suitable for use in oral care compositions include edible polyhydric alcohols such as glycerol, sorbitol, xylitol, butylene glycol, polyethylene glycol, propylene glycol, mannitol, and lactitol. Humectants help keep oral care compositions, such as pastes, from hardening upon exposure to air, give oral care compositions a moist feel to the mouth, and may impart desirable sweetness.

Surfactants include anionic, nonionic, amphoteric, zwitterionic and cationic synthetic detergents. Anionic surfactants include the water-soluble salts of alkyl sulfates having 8-20 carbon atoms in the alkyl radical (such as sodium alkyl sulfate), the water-soluble salts of sulfonated monoglycerides of fatty acids having from 8-20 carbon atoms (such as sodium lauryl sulfate and sodium coconut monoglyceride sulfonates), sarcosinates (such as sodium and potassium salts of lauroyl sarcosinate, myristoyl sarcosinate, palmitoyl sarcosinate, stearoyl sarcosinate and oleoyl sarcosinate), taurates, higher alkyl sulfoacettes (such as sodium lauryl sulfoacetate), isethionates (such as sodium lauroyl isethionate), sodium laureth carboxylate, sodium dodecyl benezesulfonate, and mixtures of the foregoing. Preferred are the sarcosinates since they inhibit acid formation in the mouth due to carbohydrate breakdown. Nonionic surfactants include poloxamers (sold under the tradename Pluronic), polyoxyethylene sorbitan esters (sold under the tradename Tween), fatty alcohol ethoxylates, polyethylene oxide condensates of alkyl phenols, products derived from the condensation of ethylene oxide with fatty acids, fatty alcohols, fatty amides, polyhydric alcohols, and polypropyleneoxide, ethylene oxide condensates of aliphatic alcohols, long-chain tertiary amine oxides, long-chain tertiary phospine oxides, long-chain dialkyl sulfoxides, and mixtures of such materials. Amphoteric surfactants include betaines (such as cocamidopropylbetaine), derivatives of aliphatic secondary and tertiary amines in which the aliphatic radical can be a straight or branched chain and wherein one of the aliphatic substituents contains about 8-18 carbon atoms and one contains an anionic water-solubilizing group (such as carboxylate, sulfonate, sulfate, phosphate or phosphonate), and mixtures of such materials. Zwitterionic surfactants include derivatives of aliphatic quaternary ammonium, phosphonium and sulfonium compounds in which the aliphatic radical can be a straight or branched chain and wherein one of the aliphatic substituents contains about 8-18 carbon atoms and one contains an anionic water-solubilizing group (such as carboxy, sulfonate, sulfate, phosphate or phosphonate). Cationic surfactants include aliphatic quaternary ammonium compounds having one long alkyl chain containing about 8-18 carbon atoms (such as lauryl trimethylammonium chloride, cetylpyridinium chloride, cetyltrimethylammonium bromide, diisobuytylphenoxyethyldimethylbenzylammonium chloride, coconut alkyltrimetylammonium nitrite, cetylpyridinium fluoride). Certain cationic surfactants can also act as antimicrobials.

Thickening agents include carboxyvinyl polymers, polyvinylpyrrolidone, polyacrylates, carrageenan, cellulose derivatives (e.g., hydroxypropyl cellulose, hydroxypropyl methyl cellulose, methyl cellulose, and hydroxyethyl cellulose), laponite, water-soluble salts of cellulose ethers (such as sodium carboxymethylcellulose and sodium carboxymethyl hydroxyethyl cellulose), natural gums (such as gum karaya, xanthan gum, gum arabic and gum tragacanth), polymeric polyether compounds (such as polyethylene oxide and polypropylene oxide), homopolymers of acrylic acid crosslinked with an alkyl ether of pentaerythritol, alkyl ether of sucrose, carbomers (sold under the tradename Carbopol®, starch, copolymers of lactide and glycolide monomers (the copolymer having an average molecular weight of about 1,000-120,000), colloidal magnesium aluminum silicate and finely divided silica. Thickening agents will be added in amounts sufficient to give a desired consistency to an oral care composition.

Abrasives include silicas (including gels and precipitates), aluminas, calcium carbonates, calcium phosphates, dicalcium phosphates, tricalcium phosphates, hydroxyapatites, calcium pyrophosphates, trimetaphosphates, insoluble polymetaphopsphates (such as insoluble sodium polymetaphosphate and calcium polymetaphosphate), magnesium carbonates, magnesium oxides, resinous abrasive materials (such as particulate condensation products of urea and formaldehyde), particulate thermosetting polymerized resins (suitable resins include melamines, phenolics, ureas, melamine-ureas, melamine-formaldehydes, urea-formaldehydes, melamine-urea-formaldehydes, cross-linked epoxides and cross-linked polyesters), and combinations of the foregoing. Silica abrasives are preferred because they provide excellent dental cleaning and polishing performance without unduly abrading tooth enamel or dentine.

Flavoring agents include peppermint, oil, spearmint oil, wintergreen oil, clove, menthol, dihydroanethole, estragole, methyl salicylate, eucalyptol, cassia, 1-menthyl acetate, sage, eugenol, parsley oil, menthone, oxanone, alpha-irisone, alpha-ionone, anise, marjoram, lemon, orange, propenyl guaethol, cinnamon, vanillin, ethyl vanillin, thymol, linalool, limonene, isoamylacetate, benzaldehyde, ethylbutyrate, phenyl ethyl alcohol, sweet birch, cinnamic aldehyde, cinnamaldehyde glycerol acetal (known as CGA), and mixtures of the foregoing.

Sweetening agents include sucrose, glucose, saccharin, dextrose, levulose, lactose, mannitol, sorbitol, fructose, maltose, xylitol, saccharin salts, thaumatin, aspartame, D-tryptophan, dihydrochalcones, acesulfame, cyclamate salts, and mixtures of the foregoing.

In addition to the flavoring and sweetening agents, the oral care compositions may include coolants, salivating agents, warming agents and numbing agents as optional ingredients. Coolants include carboxamides, menthol, paramenthan carboxamides, isopropylbutanamide, ketals, diols, 3-1-menthoxypropane-1,2-diol, menthone glycerol acetal, menthyl lactate, and mixtures thereof. Salivating agents include Jambu® (manufactured by Takasago). Warming agents include capsicum and nicotinate esters (such as benzyl nicotinate). Numbing agents include benzocaine, lidocaine, clove bud oil and ethanol.

Antibacterial and anti-plaque agents include triclosan, sanguinarine and sanguinaria, quaternary ammonium compounds, cetylpyridinium chloride, tetradecylpyridinium chloride and N-tetradecyl-4-ethylpyridinium chloride, benzalkonium chloride, bisquanides, chlorhexidine, chlorhexidine digluconate, hexetidine, octenidine, alexidine, halogenated bisphenolic compounds, 2,2′-methylenebis-(4-chloro-6-bromophenol), 5-chloro-2-(2,4-dichlorophenoxy)-phenol, salicylanilide, domiphen bromide, delmopinol, octapinol, other piperadino derivatives, nicin, zinc stannous ion agents, antibiotics (such as augimentin, amoxicillin, tetracycline, doxydcycline, minocycline, and metronidazole), analogs and salts of the foregoing, and mixtures of the foregoing.

Anti-caries agents include sodium fluoride, stannous fluoride, potassium fluoride, amine fluorides, indium fluoride, sodium monofluorophosphate, calcium lactate, calcium glycerophosphates, strontium salts, and strontium polyacrylates.

Anti-calculus agents include pyrophosphate salts such as dialkali metal pyrophosphate salts and tetraalkali metal pyrophosphate salts (e.g., disodium dihydrogen pyrophosphate, tetrasodium pyrophosphate and tetrapotassium pyrophosphate, in their hydrated and unhydrated forms). Other anti-calculus agents which can be used instead of, or in addition to, the pyrophosphate salts include synthetic anionic polymers (such as polyacrylates and copolymers of maleic anhydride or acid and methyl vinyl ether), polyaminopropane sulfonic acid, zinc citrate trihydrate, polyphosphates (such as tripolyphosphate and hexametaphosphate), polyphosphonates (such as disodium ethane-1-hydroxy-1,1-diphosphonate (EHDP), methanedisphosphonic acid, and 2-phosphonobutane-1,2,4-tricarboxylic acid), and polypeptides (such as polyaspartic acid and polyglutamic acid).

The pH of the oral compositions of the invention should not be acidic, since acidic conditions will lessen the effectiveness of the non-peptide polyamine chelating agent and physiologically-acceptable salts thereof. Thus, the pH of the oral care compositions of the invention should be greater than about 6.5, preferably from about 7.0 to about 8.5, more preferably from about 7.2 to about 7.6. Thus, a pH-adjusting agent and/or a buffering agent or agents may need to be included in the oral care compositions. The pH-adjusting agent may be any compound or mixture of compounds that will achieve the desired pH. Suitable pH-adjusting agents include organic and inorganic acids and bases, such as benzoic acid, citric acid, potassium hydroxide, and sodium hydroxide. Buffering agents include acetate salts, borate salts, carbonate salts, bicarbonate salts (e.g., an alkali metal bicarbonate, such as sodium bicarbonate (also known as baking soda)), gluconates, tartrates, sulfates, citrates (such as sodium citrate), benzoate salts, nitrate salts (such as sodium and potassium nitrate), phosphate salts (such as potassium and sodium phosphate), and combinations of the foregoing as needed to achieve and maintain the desired pH.

The oral care compositions of the invention may further include one or more antioxidants, anti-inflammatory compounds, and/or metal-binding compounds in addition to a non-peptide polyamine chelating agent or a physiologically-acceptable salt thereof.

Suitable anti-inflammatory agents include ibuprofen, flurbiprofen, ketoprofen, aspirin, kertorolac, naproxen, indomethacin, piroxicam, meclofenamic acid, steroids, and mixtures of the foregoing.

Suitable antioxidants include superoxide dismutase, catalase, glutathione peroxidase, ebselen, glutathione, cysteine, N-acetyl cysteine, penicillamine, allopurinol, oxypurinol, ascorbic acid, α-tocopherol, Trolox (water-soluble α-tocopherol), vitamin A, β-carotene, fatty-acid binding protein, fenozan, probucol, cyanidanol-3, dimercaptopropanol, indapamide, emoxipine, dimethyl sulfoxide, and others. See, e.g., Das et al., Methods Enzymol., 233, 601-610 (1994); Stohs, J. Basic Clin. Physiol. Pharmacol., 6, 205-228 (1995).

Suitable metal-binding compounds include metal-binding peptide and/or non-peptide chelators, such as those described in PCT applications WO 01/25265 and WO 02/64620 and co-pending U.S. application Ser. No. 10/186,168, filed Jun. 27, 2002, the complete disclosures of which are incorporated herein by reference, and references cited in these three applications. Other suitable metal-binding compounds are known in the art.

The oral care compositions of the invention may advantageously contain an enzyme inhibitor for an additional therapeutic effect. For instance, certain proteases are involved in inflammatory processes and others have been implicated in tissue breakdown in the mouth. Suitable protease inhibitors include metalloproteinase and serine protease inhibitors, such as those described in U.S. Pat. Nos. 6,403,633, 6,350,438, 6066673, 5,622,984, and 4,454,338, the complete disclosures of which are incorporated herein by reference. Further, a collegenase inhibitor, such as Periostat® low-dose doxycyline (CollaGenex), could be included.

Many other ingredients are known that may be incorporated into oral care compositions. These include suspending agents (such as a polysaccharide—see U.S. Pat. No. 5,466,437), polymeric compounds which can enhance the delivery of active ingredients (such as copolymers of polyvinylmethylether with maleic anhydride and those delivery enhancing polymers described in DE 942,643 and U.S. Pat. No. 5,466,437), oils, waxes, silicones, coloring agents (such as FD&C dyes), color change systems, preservatives (such as methylparaben, propylparaben, and sodium benzoate), opacifying agents (such as titanium dioxide), plant extracts, solubilizing agents (such as propylene glycol; see, e.g., U.S. Pat. No. 5,466,437), enzymes (such as dextranase and/or mutanase, amyloglucosidase, glucose oxidase with lactoperoxidase, and neuraminidases), synthetic or natural polymers, tooth whitening agents (such from about 0.1% to about 10% by weight of a peroxygen compound; see additional discussion of tooth whitening compositions below), an alkali metal bicarbonate (such as sodium bicarbonate (also known as baking soda), generally present at from about 0.01% to about 30% by weight), desensitizers (such as potassium salts (e.g., potassium nitrate, potassium citrate, potassium chloride, potassium tartrate, potassium bicarbonate, and potassium oxalate), and strontium salts), analgesics (such as lidocaine or benzocaine), anti-fungal agents, antiviral agents, etc.

The presence of a significant amount of copper and iron salts is preferably avoided. The presence of significant amounts of copper and iron ions in the oral care compositions could reduce the ability of the trientine or a physiologically-acceptable salt thereof to bind copper and iron ions found in the mouth.

It will be appreciated that a wide variety of different oral care compositions can be prepared utilizing the above described ingredients and other ingredients known in the art or which will be developed. It is within the skill in the art to chose appropriate ingredients and combinations of ingredients and to determine an effective amount of a non-peptide polyamine chelating agent or a physiologically-acceptable salt thereof to include in a particular oral care composition, given the knowledge in the art and the guidance provided herein.

What follows are a few examples of oral care compositions into which a non-peptide polyamine chelating agent or a physiologically-acceptable salt thereofcould be incorporated. It will be understood by those skilled in the art that additional types of oral care compositions and additional oral care compositions having different ingredients and/or different amounts of ingredients can be prepared utilizing the knowledge and skill in the art and the guidance provided herein.

Dentrifices include toothpastes, tooth gels, tooth powders and liquid dentrifices. Toothpastes and tooth gels generally include a dental abrasive, a surfactant, a thickening agent, a humectant, a flavoring agent, a sweetening agent, a coloring agent and water. Toothpastes and tooth gels may also include opacifying agents, anti-caries agents, anti-calculus agents, tooth whitening agents, and other optional ingredients. Typically, a toothpaste or tooth gel will contain from about 5% to about 70%, preferably from about 10% to about 50%, of an abrasive, from about 0.5% to about 10% of a surfactant, from about 0.1% to about 10% of a thickening agent, from about 10% to about 80% of a humectant, from about 0.04% to about 2% of a flavoring agent, from about 0.1% to about 3% of a sweetening agent, from about 0.01% to about 0.5% of a coloring agent, from about 0.05% to about 0.3% of an anti-caries agent, from about 0.1% to about 13% of an anti-calculus agent, and from about 2% to about 45% water. Tooth powders of course contain substantially all non-liquid components and typically contain from about 70% to about 99% abrasive. Liquid dentrifices may comprise water, ethanol, a humectant, a surfactant, a thickening agent, an abrasive (if an abrasive is included, a suspending agent (e.g., a high molecular weight polysaccharide) must be included; see U.S. Pat. No. 5,466,437), an antibacterial agent, an anti-caries agent, a flavoring agent and a sweetening agent. A typical liquid dentrifice will comprise from about 50% to about 85% water, from about 0.5% to about 20% ethanol, from about 10% to about 40% of a humectant, from about 0.5% to about 5% of a surfactant, from about 0.1% to about 10% of a thickening agent, and may contain from about 10% to about 20% of an abrasive, from about 0.3% to about 2% of a suspending agent, from about 0.05% to about 4% of an antibacterial agent, from about 0.0005% to about 3% of an anti-caries agent, from about 0.1% to about 5% of a flavoring agent, and from about 0.1% to about 5% of a sweetening agent.

Gels include dentrifice gels (see description above), non-abrasive gels and subgingival gels. Non-abrasive gels and subgingival gels generally include a thickening agent, a humectant, a flavoring agent, a sweetening agent, a coloring agent, and water. Such gels may also include one or more anti-caries agents and/or anti-calculus agents. Typically, such a gel will contain from about 0.1% to about 20% of a thickening agent, from about 10% to about 55% of a humectant, from about 0.04% to about 2% of a flavoring agent, from about 0.1% to about 3% of a sweetening agent, from about 0.01% to about 0.5% of a coloring agent, and the balance water. Such gels may also contain from about 0.05% to about 0.3% of an anti-caries agent and from about 0.1% to about 13% of an anti-calculus agent.

Creams generally include a thickening agent, a humectant and a surfactant, and may include a flavoring agent, a sweetening agent, a coloring agent. Typically, a cream will contain from about 0.1% to about 30% of a thickening agent, from about 0% to about 80% of a humectant, from about 0.1% to about 5% of a surfactant, from about 0.04% to about 2% of a flavoring agent, from about 0.1% to about 3% of a sweetening agent, from about 0.01% to about 0.5% of a coloring agent, and from about 2% to about 45% of water.

Ointments suitable for oral use are described in, e.g., U.S. Pat. Nos. 4,847,283, 5,855,872 and 5,858,408, the complete disclosures of which are incorporated herein by reference. Ointments generally include one or more of the following: fats, oils, waxes, parafins, silicones, plastibase, alcohols, water, humectants, surfactants, thickening agents, talc, bentonites, zinc oxide, aluminum compounds, preservatives, antiviral compounds, and other ingredients. For instance, the ointment may comprise from about 80% to about 90% petrolatum and from about 10% to about 20% ethanol or propylene glycol. As another example, the ointment may comprise about 10% petrolatum, about 9% lanolin, about 8% talc, about 32% cod liver oil, and about 40% zinc oxide. As a third example, the ointment may comprise from about 30% to about 45% water, from about 10% to about 30% oil (e.g.,petrolatum or mineral oil), from about 0.1% to about 10% emulsifier (e.g., wax NF), from about 2% to about 20% humectant (e.g., propylene glycol), from about 0.05% to about 2% preservatives (e.g., methyl paraben and propyl paraben), and from about 10% to about 40% sterol alcohol.

Mouthwashes, rinses, gargles and sprays generally include water, ethanol, and/or a humectant, and preferably also include a surfactant, a flavoring agent, a sweetening agent, and a coloring agent, and may include a thickening agent and one or more anti-caries agents and/or anti-calculus agents. A typical composition contains from about 0% to about 80% of a humectant, from about 0.01% to about 7% of a surfactant, from about 0.03% to about 2% of a flavoring agent, from about 0.005% to about 3% of a sweetening agent, from about 0.001% to about 0.5% of a coloring agent, with the balance being water. Another typical composition contains from about 5% to about 60%, preferably from about 5% to about 20%, ethanol, from about 0% to about 30%, preferably from about 5% to about 20%, of a humectant, from about 0% to about 2% emulsifying agents, from about 0% to about 0.5% of a sweetening agent, from about 0% to about 0.3% of a flavoring agent, and the balance water. A further typical composition contains from about 45% to about 95% water, from about 0% to about 25%, ethanol, from about 0% to about 50% of a humectant, from about 0.1% to about 7% of a surfactant, from about 0.1% to about 3% of a sweetening agent, from about 0.4% to about 2% of a flavoring agent, and from about 0.001% to about 0.5% of a coloring agent. These compositions may also comprise from about 0.05% to about 0.3% of an anti-caries agent, and from about 0.1% to about 3% of an anti-calculus agent

Solutions generally include water, a preservative, a flavoring agent, and a sweetening agent, and may include a thickening agent and/or a surfactant. Typically, solutions contain from about 85% to about 99% water, from about 0.01% to about 0.5% of a preservative, from about 0% to about 5% of a thickening agent, from about 0.04% to about 2% of a flavoring agent, from about 0.1% to about 3% of a sweetening agent, and from about 0% to about 5% of a surfactant.

Among the preferred oral care compositions are mouthwashes, rinses, gargles, sprays and solutions comprising trientine. Particularly preferred are mouthwashes, rinses, gargles, sprays and solutions comprising trientine at a concentration of from about 1.0 μM to about 1.0 mM, preferably from about 10 μM to about 750 μM, more preferably from about 50 μM to about 500 μM, and most preferably from about 200 μM to about 300 μM.

Lozenges and mints generally include a base, a flavoring agent and a sweetening agent. The base may be a candy base (hard sugar candy), glycerinated gelatin or a combination of sugar with sufficient mucilage to give it form. See U.S. Pat. No. 6,350,438 and Remington, The Science And Practice Of pharmacy, 19^{th} edition (1995). Lozenge compositions also typically include one or more fillers (e.g., a compressible sugar) and lubricants.

Chewing gums, chewable tablets and chewable lozenges are described in U.S. Pat. Nos. 6,471,991, 6,296,868, 6,146,661, 6,060,078, 5,869,095, 5,709,873, 5,476,647, and 5,312,626, PCT applications WO 84/04453 and WO 99/02137, and Lieberman et al., Pharmaceutical Dosage Forms, 2^{nd} ed. (1990), the complete disclosures of which are incorporated here in by reference.

As one example, a compressed chewable tablet comprises a water-disintegratable, compressible carbohydrate (such as mannitol, sorbitol, maltitol, dextrose, sucrose, xylitol, lactose and mixtures thereof), a binder (such as cellulose, cellulosic derivatives, polyvinyl pyrrolidone, starch, modified starch and mixtures thereof), and, optionally, a lubricant (such as magnesium stearate, stearic acid, talc, and waxes), sweetening, coloring and flavoring agents, a surfactant, a preservative, and other ingredients. All of the ingredients, including the non-peptide polyamine chelating agent, or a physiologically-acceptable salt thereof, are dry blended and compressed into a tablet.

As another example, a chewable tablet may comprise a core surrounded by an outer layer wrapping the core. The core may comprise a non-peptide polyamine chelating agent or a physiologically-acceptable salt thereof and, optionally, other active ingredients in ajelly base or a chewable base. The outer layer may be a chewable base. The jelly base may comprise pectin, sorbitol, maltitol, isomalt, liquid glucose, sugar, citric acid and/or a flavoring agent. The chewable base of the core or outer layer may be a gum, soft candy, nougat, caramel or hard candy. The tablets are formed by extrusion of the core and outer layer to form a rope, followed by cutting the rope into tablets.

Chewing gum compositions generally include a gum base, a flavoring agent and a sweetening agent. Suitable gum bases include jelutong, rubber, latex, chicle, and vinylite resins, desirably with conventional plasticizers or softeners. Plasticizers include triacetin, acetyl tributyl citrate, diethyl sebacetate, triethyl citrate, dibutyl sebacetate, dibutyl succinate, diethyl phthalate and acetylated monoglycerides. Typically, chewing gum compositions contain from about 50% to about 99% gum base, from about 0.4% to about 2% of a flavoring agent and from about 0.01% to about 20% of a sweetening agent. The a non-peptide polyamine chelating agent or a physiologically-acceptable salt thereof, and other active ingredients may be incorporated into a gum base by, e.g., stirring them into a warm gum base or coating them onto the outer surface of the gum base.

Films and sheets, and gels which form solids in the mouth, made of lactide/glycolide copolymers are described in U.S. Pat. Nos. 5,198,220, 5,242,910 and 6.350,438. Another polymer film suitable for use in the mouth is described in PCT application WO 95/32707. All of these materials slowly release active agents contained in them into the mouth. Other compositions (including pastes, gels, ointments, liquids and films) providing for slow release of active agents are also known. See, e.g., U.S. Pat. Nos. 5,032,384, 5,298,237, 5,466,437, 5,709,873, and 6,270,781.

Tooth whitening compositions will comprise a tooth whitening agent. Tooth whitening agents include peroxides, percarbonates and perborates of the alkali and alkaline earth metals or complex compounds containing hydrogen peroxide. Tooth whitening agents also include peroxide salts of the alkali or alkaline earth metals. The most commonly used tooth whitening agent is carbamide peroxide. Other commonly used tooth whitening agents are hydrogen peroxide, peroxyacetic acid and sodium perborate. These tooth whitening agents liberate active oxygen and hydrogen peroxide. Tooth whitening agents can be present in tooth whitening compositions at a concentration of from about 0.1% to about 90%; typically, the concentration of carbamide peroxide in tooth whitening compositions is from about 10% to about 25%.

Many tooth whitening compositions are known in the art, including aqueous solutions, gels, pastes, liquids, films, strips, one-part systems, two-part systems, compositions that require activation of the tooth whitening agent (e.g., by inclusion of a radiant-energy or heat-energy absorbing substance, such as substantially conjugated hydrocarbons, which activates the bleaching agent when irradiated), etc. See, e.g., U.S. Pat. Nos. 5,302,375, 5,785,887, 5,858,332, 5,891,453, 5,922,307, 6,322,773, 6,419,906, and PCT applications WO 99/37236, WO 01/89463 and WO 02/07695, the complete disclosures of which are incorporated herein by reference. Also, many other oral care compositions (e.g., toothpastes) and devices (e.g., dental flosses) comprise a tooth whitening agent.

The use of tooth whitening compositions, or of one of the many oral care compositions and devices which comprise a tooth whitening agent, results in the production of ROS and can cause inflammation of the tissues of the mouth. Incorporation of a non-peptide polyamine chelating agent or a physiologically-acceptable salt thereof in tooth whitening compositions or other oral care compositions and devices comprising a tooth whitening agent will reduce or prevent the inflammation and/or the production of ROS. The inclusion of a non-peptide polyamine chelating agent or a physiologically-acceptable salt thereof in such compositions may also result in more effective whitening, since hydrogen peroxide, which is responsible for the whitening of teeth by the hydrogen peroxide-type whitening agents, will not be converted into hydroxyl radicals and will, therefore, remain active longer. Alternatively, an oral care composition or device comprising a non-peptide polyamine chelating agent or a physiologically-acceptable salt thereof can be used before or after the tooth whitening composition or oral care composition or device comprising a tooth whitening agent to reduce or prevent the inflammation and/or the production of ROS.

For instance, teeth are commonly whitened by applying a tooth whitening composition to the teeth by means of a dental tray or trough. A non-peptide polyamine chelating agent or a physiologically-acceptable salt thereof could be incorporated into the tooth whitening composition that is used in the tray or trough. Alternatively, a separate composition comprising a non-peptide polyamine chelating agent or a physiologically-acceptable salt thereof could be applied to the teeth in a cleaned or different tray or trough after the application of the tooth whitening composition is completed. In a further alternative, a wash or rinse comprising or a physiologically-acceptable salt or a physiologically-acceptable salt thereof could be used to rinse the mouth before and/or after the application of the tooth whitening composition.

A recently developed product for applying a tooth whitening composition to the teeth is a flexible strip. See, e.g., U.S. Pat. Nos. 5,891,453 and 6,419,906. A a non-peptide polyamine chelating agent or a physiologically-acceptable salt thereof could be incorporated into such strips. For instance, a non-peptide polyamine chelating agent or a physiologically-acceptable salt thereof could be incorporated into the tooth whitening composition, which is then applied to the strips, or a solution, gel or other composition comprising the non-peptide polyamine chelating agent or a physiologically-acceptable salt thereof could be separately applied to the strips, either during their manufacture or just prior to use by the patient. In yet another alternative, strips comprising a tooth whitening composition and strips comprising the non-peptide polyamine chelating agent or a physiologically-acceptable salt thereof could both be supplied to the patient and would be used sequentially.

As noted above, preliminary data indicate that trientine may be effective in whitening heavily stained teeth. Accordingly, it is possible that it can be used alone to whiten teeth, and its use as described in the preceding two paragraphs may contribute to the whitening of teeth by known tooth whitening agents, whether used before, with or after such agents.

The oral care compositions of the invention may comprise a single phase or a plurality of phases. A plurality of phases will be used, e.g., where some of the ingredients are incompatible, some of the ingredients are unstable, or the ingredients are best combined at the time of use. Thus, one of the phases will include some of the ingredients, and the remainder of the ingredients will be contained in one or more additional phases. The plurality of phases may be a plurality of separate compositions, in which case the plurality of phases will be provided in a plurality of separate containers or in a plurality of compartments in a single container, and the plurality of phases will be combined at the time of use. As an alternative the plurality of phases may be formed by encapsulating some of the ingredients, in which case the plurality of phases may all be contained in a single container. Multi-phase oral care compositions are described in, e.g., U.S. Pat. Nos. 5,302,375, 5,906,811, 5,976,507, 6,228,347 and 6,350,438 and PCT application number WO 99/37236.

The invention also provides oral care devices comprising a non-peptide polyamine chelating agent or a physiologically-acceptable salt thereof. Oral care devices of the invention include devices intended for use by consumers and patients and devices intended for use by dental professionals (e.g., dental hygienists, dentists and oral surgeons).

The oral care devices of the invention include surgical materials (such as sutures and sponges), flosses, tapes, chips, strips, fibers, a toothpick or rubber tip, syringes, dental implants and dental appliances (such as trays and troughs that fit over and cover the teeth and, optionally, the periodontal tissue) having a non-peptide polyamine chelating agent or a physiologically-acceptable salt thereof adhered to, absorbed into, bound to, attached to, entrapped in, enclosed in, coated onto, or otherwise incorporated into, them. See, e.g., U.S. Pat. Nos. 5,709,873, 5,863,202, 5,891,453, 5,967,155, 5,972,366, 5,980,249, 6,026,829, 6,080,481, 6,102,050, 6,350,438, 6,419,906, PCT application WO 02/13775, and EP application 752833, which describe such oral care devices and methods of incorporating compounds into them (the complete disclosures of all of these patents and applications are incorporated herein by reference). For instance, a non-peptide polyamine chelating agent or a physiologically-acceptable salt thereof can be incorporated into a binder (e.g., a wax or polymer) and coated onto dental floss, dental floss can be soaked in a bath of a liquid containing a non-peptide polyamine chelating agent or a physiologically-acceptable salt thereof to impregnate or coat the floss with the non-peptide polyamine chelating agent or a physiologically-acceptable salt thereof, a non-peptide polyamine chelating agent or a physiologically-acceptable salt thereof in solid form can be incorporated into a polymer film suitable for application to the teeth, a non-peptide polyamine chelating agent or a physiologically-acceptable salt thereof in a solution or gel can be applied to a flexible strip suitable for application to teeth, or a suture or other surgical material can be soaked in a solution containing a non-peptide polyamine chelating agent or a physiologically-acceptable salt thereof followed by removal of the solvent so that the non-peptide polyamine chelating agent or a physiologically-acceptable salt thereof becomes associated with (bound to, entrapped in, coated onto, etc.) the suture or surgical material. See, e.g., U.S. Pat. Nos. 5,891,453, 5,967,155, 5,972,366, 6,026,829, 6,080,481, 6,102,050, and 6,419,906.

Also included within the scope of the invention are oral care products for animals, such as foods, chews, and toys. Suitable products are described in U.S. Pat. No. 6,350,438.

A non-peptide polyamine chelating agent or a physiologically-acceptable salt thereof can be used to treat a tissue of an animal's mouth. “Mouth” is used herein to mean the cavity bounded externally by the lips and internally by the pharynx that encloses the tongue, gums and teeth. Thus, the tissues of the mouth include the lips, tongue, gums, buccal tissue, palate and teeth. A single tissue, a plurality of tissues, a portion of one or more tissues, all or substantially all of the tissues of the mouth, or combinations of the foregoing, may be treated according to the invention. “Treat” and variations thereof are used herein to mean to cure, ameliorate, alleviate, inhibit, prevent, reduce the likelihood of, or reduce the severity of, a disease or condition, or of at least some of the symptoms or effects thereof.

To treat a tissue of the mouth, the tissue is contacted with the non-peptide polyamine chelating agent or a physiologically-acceptable salt thereof. For instance, the tissue may be contacted with an oral care composition comprising a non-peptide polyamine chelating agent or a physiologically-acceptable salt thereof. Methods of contacting tissues of the mouth with oral care compositions are well known in the art. Suitable methods include rinsing the tissue with a solution (e.g., a mouthwash, rinse, spray, liquid dentrifice, or other solution), brushing the teeth with a dentrifice (e.g., a toothpaste, tooth gel, or powder), applying a non-abrasive solution, gel, paste, cream or ointment directly to the tissue (with or without the use of an applicator), chewing gum, chewing or sucking a lozenge, mint or tablet, and many other means oftopical application. Suitable applicators for applying oral care compositions, such as solutions, gels, pastes, creams and ointments, to a tissue include a swab, a stick, a plastic paddle, a dropper, a syringe, a strip (such as those described in U.S. Pat. Nos. 5,891,453 and 6,419,906), a finger, or a dental tray or appliance (such as those shown in U.S. Pat. Nos. 5,863,202 and 5,980,249 and EP application 752833) which allows for immersion of the teeth and, optionally, the periodontal tissue in, e.g., a gel or solution. In addition, to treat a tissue of the mouth, the tissue may be contacted with an oral care device comprising a non-peptide polyamine chelating agent or a physiologically-acceptable salt thereof. Methods of contacting tissues of the mouth with oral care devices are well known in the art. For instance, sutures can be used to close a surgical wound or a wound resulting from a tooth extraction, dental floss can be used to floss the teeth, etc.

The treatment of the tissue can be prophylactic treatment. For instance, the tissue may be treated as part of a prophylactic oral care regimen. The non-peptide polyamine chelating agent or a physiologically-acceptable salt thereof can be incorporated into an oral care composition or device, such as a toothpaste, a tooth gel, a mouthwash or rinse, or a dental floss, that is employed in such a regimen and will be used preferably at least once per day, more preferably two or three times per day. In another alternative, the non-peptide polyamine chelating agent or a physiologically-acceptable salt thereof may be contained in a separate oral care composition or device which will be used separately from other compositions and devices employed in the prophylactic oral care regimen. For instance, the non-peptide polyamine chelating agent or a physiologically-acceptable salt thereof can be incorporated into a mouthwash or rinse, a gum, a lozenge or a chewable tablet, which would preferably be used at least once per day, more preferably at least two or three times per day. It may be particularly beneficial for those patients who utilize tobacco products to use a non-peptide polyamine chelating agent or a physiologically-acceptable salt thereof as part of a prophylactic oral care regimen to attempt to ameliorate the damage done to tissues of the mouth by such products.

It is known to include metal salts, particularly copper salts, in toothpastes and other oral care compositions, generally as antibacterial, anti-plaque, anti-caries, and anti-gingivitis agents. See, e.g., U.S. Pat. Nos. 5,286,479, 5,298,237, and 6,355,706, EP application 658,565, PCT application WO 92/08441, Japanese application 41 59211, Waerhaug et al., J. Clin. Periodontol., 11:176-180 (1984). The use of oral care compositions containing copper salts could be harmful to the tissues of the mouth, since free copper ions catalyze the formation of ROS. Thus, the use of an oral care composition of the present invention at an appropriate time after the use of the copper-containing compositions (i. e., allowing sufficient time for the copper salts to exert their activity) could be very beneficial in reducing the damage done by ROS generated by copper ions present in the mouth as a result of use of these products. For instance, the non-peptide polyamine chelating agent or a physiologically-acceptable salt thereof could conveniently be supplied in a gum, lozenge or chewable table which would be chewed or sucked after use of the copper-containing compositions.

Tissues may also be treated prophylactically in connection with a variety of dental procedures, including surgeries and tooth extractions. For instance, the tissue(s) on which surgery is being performed, those tissues near the area where the surgery is being performed or, for ease of treatment, all or substantially of the tissues of the mouth, can be treated prior to surgery, during surgery, after the surgery, or combinations thereof. Similarly for a tooth extraction, the tissue(s) surrounding the tooth which is to be extracted, adjacent tissues or, for ease of treatment, all or substantially of the tissues of the mouth, can be treated prior to tooth extraction, during the tooth extraction, after the tooth extraction, or combinations thereof. For instance, the mouth could be rinsed prior to surgery or tooth extraction with a solution comprising a non-peptide polyamine chelating agent or a physiologically-acceptable salt thereof, the wound(s) caused by the surgery or tooth extraction could be closed with sutures having a non-peptide polyamine chelating agent or a physiologically-acceptable salt thereof incorporated into them, and/or the mouth could be rinsed immediately after the surgery or tooth extraction, and/or at intervals thereafter, with a solution comprising a non-peptide polyamine chelating agent or a physiologically-acceptable salt thereof.

Tissues can also be treated prophylactically in connection with radiation, such as dental x-rays. Finally, as described above, tissues may be treated prophylactically in connection with the whitening of the teeth of an animal.

A non-peptide polyamine chelating agent or a physiologically-acceptable salt thereof can be used to treat a disease or condition of a tissue of an animal's mouth. Diseases and conditions treatable according to the invention include inflammation and inflammatory disease and conditions, such as gingivitis and periodontitis, and any disease or condition involving, caused by, or exacerbated by, ROS.

It is understood by those skilled in the art that the dosage amount of a non-peptide polyamine chelating agent or a physiologically-acceptable salt thereofneeded to treat a tissue of an animal's mouth will vary with the particular the type of oral care composition employed, whether the treatment is prophylactic or for the treatment of a disease or condition, the identity of the disease or condition to be treated, the severity of the disease or condition, the duration of the treatment, the identify of any other drugs being administered to the animal, the age, size and species of the animal, and like factors known in the medical and veterinary arts. In general, a suitable daily dose of a non-peptide polyamine chelating agent or a physiologically-acceptable salt thereofwill be that amount of the compound which is the lowest dose effective to produce a therapeutic effect. It is expected that usage of oral care compositions comprising from about 0.001% to about 25%, preferably from about 2.5% to about 12.5%, most preferably from about 5.0% to about 6.0%, by weight of a non-peptide polyamine chelating agent or a physiologically-acceptable salt thereof one or more times per day will provide effective daily dosages. However, the actual daily dosage to be employed, the number of treatments per day, and the length of treatment will be determined by an attending physician, dentist or veterinarian within the scope of sound medical judgment.

The invention also provides a kit comprising an oral care product according to the invention. In the case where the oral care product is an oral care composition, the kit may also include an applicator for applying the oral care composition to a tissue of an animal's mouth, such as a swab, a stick, a plastic paddle, a dropper, a syringe, a strip (such as that described in U.S. Pat. Nos. 5,891,453 and 6,419,906) or a dental tray or appliance (such as those shown in U.S. Pat. Nos. 5,863,202 and 5,980,249 and EP application 752833) which allows for immersion of the teeth and, optionally, the periodontal tissue in, e.g., a gel or solution. The kit could also include a cup, vial or other device for dispensing and/or measuring the amount of the oral care composition of the invention needed for the intended use. Of course, the kits could include both an oral care composition and an oral care device according to the invention. In addition to an oral care composition and/or device of the invention, the kits could also comprise another type of oral care composition or device, such as a tooth whitening composition, strips comprising a tooth whitening agent, applicators for applying oral care compositions, etc. Kits according to the invention will also include instructions for using the kit and/or the oral care product of the invention and may include any other desired items.

It is to be noted that “a” or “an” entity refers to one or more of that entity. For example, “a cell” refers to one or more cells.

### EXAMPLES

### Example 1

### Inhibition of IL-8 Release

Interleukin 8 (IL-8) is a pro-inflammatory cytokine and a potent chemoattractant and activator of neutrophils. It has also been reported to be a chemoattractant and activator of T-lymphocytes and eosinophils. IL-8 is produced by immune cells (including lymphocytes, neutrophils, monocytes and macrophages), fibroblasts and epithelial cells. Reports indicate an important role for IL-8 in the pathogenesis of many inflammatory disorders, including gingivitis and periodontal disease.

It has recently been found that endothelial cells secrete markedly elevated levels of IL-8 after exposure to a physiologically relevant concentration of copper (see co-pending U.S. application Ser. No. 10/186,168, filed Jun. 27, 2002, now published U.S. application No. US20030130185, published Jul. 10, 2003, and Bar-Or, Thomas, Yukl, Rael, Shimonkevitz, Curtis and Winkler, “Copper Stimulates the Synthesis and Release of Interleukin-8 in Human Endothelial Cells: A Possible Early Role in Systemic Inflammatory Responses,” Shock, 20(2):154-158 (August 2003); also see PCT WO 03/043518, published May 30, 2003). This example investigates the effect of the addition of trientine on copper-induced IL-8 secretion from endothelial cells.

Human umbilical vein endothelial cells (HUVEC) were grown to confluence on tissue culture plates (Griener) in endothelial cell basal medium-2 (EGM₂ medium) (Cambrex) at 37° C. and 10% CO₂. Then, the cells were washed two times with 37° C. serum-free medium (EGM₂ medium without serum and ascorbic acid supplemented with ITSS (insulin transferrin sodium selenite medium supplement) (Sigma)) and were subsequently treated for 24 hours with serum-free medium containing (i) 50 μM CuCl₂, (ii) 50 μM, 100 μM, 150 μM or 250 μM Syprine® trientine hydrochloride (Merck & Co., Inc.), or (iii) both of them (n=3, in duplicate). Syprine® trientine hydrochloride (N,N′-bis(2-aminoethyl)-1,2-ethanediamine dihydrochloride) is a drug currently approved for removal of excess copper in Wilson's disease by oral administration to patients suffering from the disease who are intolerant of penicillamine (Merck & Co., Inc. publication 7664604, issued January 2001). It is sold as capsules, and the contents of one capsule were dissolved in culture medium to give a solution containing 100 mM trientine hydrochloride. After the incubation with CUCl₂ and/or Syprine® trientine hydrochloride, medium was removed from each of the cells and analyzed for IL-8 by ELISA.

The IL-8 ELISA was performed as follows. Anti-human IL-8 antibody (Pierce Endogen, Rockford, Ill.; catalogue number M801-E, lot number CK41959) was diluted to 1 μg/ml in phosphate buffered saline, pH 7.2-7.4, and 100 μl of the diluted antibody was added to each well of Nunc Maxisorb ELISA strip plates. The plates were incubated overnight at room temperature. The liquid was aspirated from the wells, and the plates were blotted on a paper towel. Then, 200 μl of assay buffer (phosphate buffered saline, pH 7.2-7.4, containing 4% bovine serum albumin (Sigma, St. Louis, Mo.; ELIS grade =low fatty acid and IgG)) were added to each well, and the plates were incubated for 1 hour at room temperature. The liquid was aspirated from the wells, and the wells were washed 3 times with wash buffer (50 mM Tris, 0.2% Tween-20, pH 7.9-8.1) and were then blotted on a paper towel. Standards and samples (50 μl/well; standards were diluted in storage buffer) were added to the wells, and the plates were incubated for 1 hour at room temperature with gentle shaking. The liquid was aspirated, the wells were washed 3 times with wash buffer, and the plates were then blotted on a paper towel. Then, 100 μl of biotin-labeled anti-human IL-8 (Pierce Endogen, Rockford, Ill.; catalogue number M802-E, lot number CE49513), diluted to 60 ng/ml in assay buffer, were added to each well. The plates were incubated for 1 hour at room temperature, the liquid was aspirated, the wells were washed 3 times with wash buffer, and the plates were blotted on a paper towel. Then, 100 μl of HRP-conjugated streptavidin (Pierce Endogen, Rockford, Ill.; catalogue number N100) in assay buffer, were added to each well. The plates were incubated for 30 minutes at room temperature, the liquid was aspirated, the wells were washed 3 times with wash buffer, and the plates were blotted on a paper towel. Finally, 100 μl of TMB substrate solution (Pierce Endogen, Rockford, Ill.; catalogue number N301) were added to each well. The plates were incubated for 30 minutes at room temperature. The reaction was stopped by adding 100 μl/well of 0.18 M H₂SO₄. The optical densities at 450 nm and 530 nm were read on an ELISA plate reader and the difference (OD 450-OD 530) calculated.

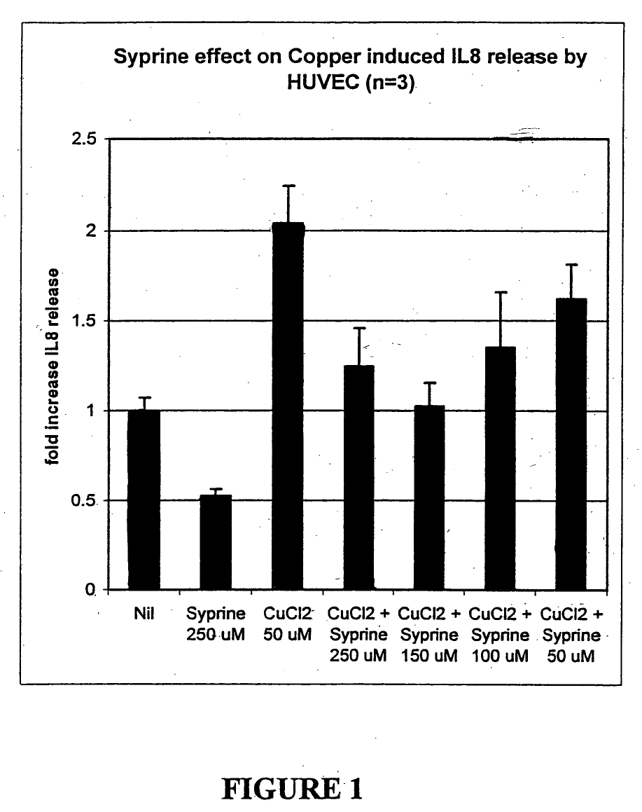

The results are shown in FIG. 1. As can be seen from FIG. 1, HUVEC incubated for 24 hours with 50 μM CuCl₂ showed a >2-fold higher IL-8 secretion as compared to controls incubated with water. At all concentrations, Syprine® trientine hydrochloride inhibited IL-8 secretion caused by CuCl₂ (compare CuCl₂ alone with CuCl₂+various concentrations of Syprine® trientine in FIG. 1). Upon visual examination, all cells appeared to be viable at 24 hours.

The results presented here provide evidence that Cu(II) ions stimulate IL-8 secretion from human endothelial cells independent of oxidative stress and that trientine, a high-affinity Cu(II)-binding compound, significantly inhibited copper-induced endothelial cell IL-8 secretion. Cu(I) ions catalyze the generation of ROS resulting in IL-8 secretion from other cell types. Since trientine binds both Cu(I) and Cu(II), it can cause a decrease in IL-8 from multiple types of cells and by two different mechanisms.

A possible mechanism for the Cu(II)-induced endothelial IL-8 secretion may be activation of serine-threonine kinase Akt (protein kinase B), which has been reported in human fibroblasts. Ostrakhovitch et al., Arch. Biochem. Biophys. 397, 232 (2002). If a similar pathway is stimulated in human endotheliium in vivo, copper could be a major contributor in the development of systemic inflammation by activating nuclear factor-kappaB (NF-kappaB). NF-kappaB is an inflammation transcription factor well known to stimulate high levels of cytokines that significantly augment vascular and cellular inflammatory responses.

### Example 2

### Effectiveness in Treating Gingivitis

A study was conducted to measure the efficacy of a low dose of trientine when used as a therapeutic mouth rinse in the treatment of gingivitis in human subjects.

A trientine-containing mouth rinse was prepared for the study by a registered pharmacist. The mouth rinse was prepared by dissolving the contents of Syprine® trientine hydrochloride (Merck & Co., Inc., USA) capsules in de-ionized water in copper-free glass containers to give a final concentration of 250 μM (55 mg/L) trientine hydrochloride.

Five adult, human volunteers were identified. The volunteers ranged in age from 18-65. There were four males and one female. None of the volunteers had smoked more than one pack of cigarettes per day within the three months prior to commencement of the study. None of the volunteers was taking medication that might interfere with copper chelation or normal inflammatory responses. None of the volunteers suffered from autoimmune diseases or diseases that might interfere with normal inflammatory or immune responses.

Oral examinations of the five volunteers were conducted by a periodontist prior to treatment. Oral pathology, number of teeth, number of sites of gingivitis, gingival index (GI), plaque index (PI), bleeding on probing (BOP) and probe depth (PD) were documented, and photographs of the teeth were taken. None of the volunteers had undergone recent oral surgery, experienced oral trauma or had gross oral pathology.

GI, PI, BOP and PD are standard terms whose meanings are well known in periodontology. In particular, PI was scored as follows:
0—No plaque.
1—A film of plaque adhering to the free gingival margin and adjacent area of the tooth. The plaque may be seen in situ only after application of disclosing solution or by using the probe on the tooth surface.
2—Moderate accumulation of soft deposits within the gingival pocket, or the tooth and gingival margin which can be seen with the naked eye.
3—Abundance of soft matter within the gingival pocket and/or on the tooth.
   GI was scored as follows:
0—No visible signs of inflammation.
1—Slight change in color and texture.
2—Noticeable inflammation and bleeding upon probing.
3—Overt inflammation and spontaneous bleeding.
   See Silness and Loe, Acta Odontol Scan, 22:292 and Loe and Silness, Acta Odontol Scan, 21:533-551.

Supragingival plaque was removed from one-half of the teeth of each volunteer, being careful not to touch the gingival margin. The volunteers were instructed to follow their normal oral hygiene routine, except that all of the volunteers were instructed to rinse, gargle and expectorate 10 ml of the trientine hydrochloride mouth rinse (containing 0.55 mg of trientine hydrochloride) for 30 seconds in the morning and at bedtime (measured dose provided), either before or after use of toothpaste. If they normally used a mouth rinse, they were instructed to substitute the trientine mouth rinse. Each of these volunteers used the trientine mouth rinse for fourteen days, at which time each of the volunteers returned to the periodontist for a repeat oral examination. The number of sites of gingivitis, GI, PI, BOP and PD were documented after fourteen days of treatment with the trientine mouth rinse, and photographs were taken.

The GI, PI, BOP and PD results are shown in Tables 1A-3C below. In these tables, “6-point” means that six measurements were taken on each tooth (inside anterior, inside face, inside posterior, outside anterior, outside face and outside posterior). “AVG” is the average of the indicated measurement for fifteen or thirty teeth, as applicable, and “STD” is the standard deviation.

It was found that the GI and BOP were decreased more than 60% and 45%, respectively, after only fourteen days of treatment with the trientine mouth rinse (see Tables 3A-C). These improvements were statistically significant (p<0.0001 and p<0.01, respectively). PI and PD were also improved (see Tables 3A-C), although the amount of improvement was not statistically significant.

The photographs of the teeth of the volunteers showed a possible improvement in whitening of some of the teeth of the volunteers as a result of the treatment with the trientine mouth rinse. In particular, the teeth of those volunteers with heavily stained teeth appeared lighter after the fourteen days of treatment with the trientine mouth rinse. However, those volunteers whose teeth showed little staining prior to treatment showed a slight darkening of their teeth after the fourteen days of treatment with the trientine mouth rinse.

Each 10 ml of the trientine mouth rinse contained 0.55 mg of trientine hydrochloride, so each patient received 1.10 mg of trientine hydrochloride per day. Syprine® trientine hydrochloride is approved by the Food & Drug Administration (FDA) for treatment of Wilson's disease with an initial recommended oral dose for an adult of 750-1250 mg/day in divided doses. Thus, each of the volunteers received about from 1/682 to about 1/1136 of the approved initial dose of trientine hydrochloride for its approved indication, and use of the low dose trientine mouth rinse significantly reduced gingivitis and periodontal bleeding on probing.

Further, comparisons of the data generated by this study with published reports of other treatments for gingivitis and periodontitis, including antimicrobial compounds (chlorhexidine) and collegenase inhibitors (low dose doxycyline hyclate), suggested that trientine is several times more effective than these other treatments and produces its effects over a shorter treatment period and with fewer side effects. In particular, chlorhexidine is the gold standard by which oral care therapeutics are judged, and comparison of the results of this study with published reports of chlorhexidine clinical trials using similar conditions and times (Caton et al., J. Clin.. Periodontol., 20:172-178 (1993); Hase et al., J. Clin. Periodontol., 22:533-539 (1995); Borrajo et al., J. Periodontol., 73:317-321 (2002)) showed trientine to consistently be superior to chlorhexidine.

TABLE 1A
PRE-TREATMENT VALUES

| | | | | TOTAL |
|---|---|---|---|---|
| | | SCALED | UNSCALED | (6-point, |
| | | (6-point, 15 | (6-point, 15 | 30 tooth |
| PATIENT | MEASUREMENT | tooth AVG) | tooth AVG) | AVG) |
| 3001 | PI | 1.6310 | 1.5595 | 1.5952 |
| 3001 | GI | 1.3929 | 1.5357 | 1.4643 |
| 3001 | BOP | 0.3810 | 0.4286 | 0.4048 |
| 3001 | PD | 2.1429 | 2.2024 | 2.1726 |
| 3002 | PI | 1.0128 | 0.6923 | 0.8526 |
| 3002 | GI | 1.4359 | 1.2692 | 1.3526 |
| 3002 | BOP | 0.3974 | 0.4359 | 0.4167 |
| 3002 | PD | 2.6667 | 2.6026 | 2.6346 |
| 3003 | PI | 0.4615 | 0.3333 | 0.3974 |
| 3003 | GI | 1.7179 | 1.7949 | 1.7564 |
| 3003 | BOP | 0.5769 | 0.4359 | 0.5064 |
| 3003 | PD | 4.8205 | 4.1154 | 4.4679 |
| 3004 | PI | 1.2500 | 0.8929 | 1.0714 |
| 3004 | GI | 1.2619 | 1.1071 | 1.1845 |
| 3004 | BOP | 0.1667 | 0.1667 | 0.1667 |
| 3004 | PD | 2.7857 | 2.6429 | 2.7143 |
| 3005 | PI | 0.1548 | 0.2262 | 0.1905 |
| 3005 | GI | 1.0120 | 1.2262 | 1.1198 |
| 3005 | BOP | 0.2976 | 0.3452 | 0.3214 |
| 3005 | PD | 1.9286 | 2.1667 | 2.0476 |

TABLE 1B
PRE-TREATMENT VALUES

| | | | | TOTAL |
|---|---|---|---|---|
| | | SCALED | UNSCALED | (6-point, |
| | | (6-point, 15 | (6-point, 15 | 30 tooth |
| PATIENT | MEASUREMENT | tooth STD) | tooth STD) | STD) |
| 3001 | PI | 0.8887 | 0.8965 | 0.8907 |
| 3001 | GI | 0.9054 | 0.8977 | 0.9017 |
| 3001 | BOP | 0.4885 | 0.4978 | 0.4923 |
| 3001 | PD | 0.9202 | 0.9541 | 0.9350 |
| 3002 | PI | 0.8753 | 0.8108 | 0.8561 |
| 3002 | GI | 0.9200 | 0.9070 | 0.9144 |
| 3002 | BOP | 0.4925 | 0.4991 | 0.4946 |
| 3002 | PD | 0.7840 | 0.9022 | 0.8431 |
| 3003 | PI | 0.5742 | 0.5736 | 0.5756 |
| 3003 | GI | 1.0799 | 0.9308 | 1.0056 |
| 3003 | BOP | 0.4972 | 0.4991 | 0.5016 |
| 3003 | PD | 1.6960 | 1.5203 | 1.6438 |
| 3004 | PI | 0.7585 | 0.7117 | 0.7548 |
| 3004 | GI | 0.5833 | 0.8217 | 0.7146 |
| 3004 | BOP | 0.3749 | 0.3749 | 0.3738 |
| 3004 | PD | 0.8370 | 0.7053 | 0.7749 |
| 3005 | PI | 0.3956 | 0.4486 | 0.4232 |
| 3005 | GI | 0.9304 | 1.0905 | 1.0167 |
| 3005 | BOP | 0.4600 | 0.4783 | 0.4684 |
| 3005 | PD | 0.7883 | 0.8041 | 0.8028 |

TABLE 2A
POST-TREATMENT VALUES

| | | | | TOTAL |
|---|---|---|---|---|
| | | SCALED | UNSCALED | (6-point, |
| | | (6-point, 15 | (6-point, 15 | 30 tooth |
| PATIENT | MEASUREMENT | tooth AVG) | tooth AVG) | AVG) |
| 3001 | PI | 1.4286 | 1.6310 | 1.5298 |
| 3001 | GI | 0.6548 | 0.7143 | 0.6845 |
| 3001 | BOP | 0.2857 | 0.2024 | 0.2440 |
| 3001 | PD | 2.2024 | 2.1786 | 2.1905 |
| 3002 | PI | 0.8333 | 0.7564 | 0.7949 |
| 3002 | GI | 0.4359 | 0.3974 | 0.4167 |
| 3002 | BOP | 0.1154 | 0.0864 | 0.1006 |
| 3002 | PD | 2.6795 | 2.5641 | 2.6218 |
| 3003 | PI | 0.4615 | 0.3590 | 0.4103 |
| 3003 | GI | 0.8846 | 0.6795 | 0.7821 |
| 3003 | BOP | 0.3718 | 0.3205 | 0.3462 |
| 3003 | PD | 4.4744 | 3.9487 | 4.2115 |
| 3004 | PI | 0.8095 | 0.9286 | 0.8690 |
| 3004 | GI | 0.2738 | 0.3810 | 0.3274 |
| 3004 | BOP | 0.0952 | 0.0714 | 0.0833 |
| 3004 | PD | 2.4702 | 2.5238 | 2.4970 |
| 3005 | PI | 0.3690 | 0.5595 | 0.4643 |
| 3005 | GI | 0.2381 | 0.3810 | 0.3095 |
| 3005 | BOP | 0.0843 | 0.0952 | 0.0898 |
| 3005 | PD | 2.0595 | 2.0357 | 2.0476 |

TABLE 2B
POST-TREATMENT VALUES

| | | | | TOTAL |
|---|---|---|---|---|
| | | SCALED | UNSCALED | (6-point, |
| | | (6-point, 15 | (6-point, 15 | 30 tooth |
| PATIENT | MEASUREMENT | tooth STD) | tooth STD) | STD) |
| 3001 | PI | 0.8400 | 0.9541 | 0.9019 |
| 3001 | GI | 0.9248 | 0.9641 | 0.9423 |
| 3001 | BOP | 0.4545 | 0.4042 | 0.4308 |
| 3001 | PD | 0.8887 | 0.8665 | 0.8752 |
| 3002 | PI | 0.7964 | 0.7926 | 0.7929 |
| 3002 | GI | 0.8310 | 0.7786 | 0.8029 |
| 3002 | BOP | 0.3216 | 0.2827 | 0.3018 |
| 3002 | PD | 0.7644 | 0.7133 | 0.7392 |
| 3003 | PI | 0.5742 | 0.5805 | 0.5778 |
| 3003 | GI | 1.0442 | 0.7976 | 0.9318 |
| 3003 | BOP | 0.4864 | 0.4697 | 0.4773 |
| 3003 | PD | 1.7035 | 1.4937 | 1.6185 |
| 3004 | PI | 0.6107 | 0.6728 | 0.6433 |
| 3004 | GI | 0.6650 | 0.6926 | 0.6790 |
| 3004 | BOP | 0.2953 | 0.2591 | 0.2772 |
| 3004 | PD | 0.8895 | 0.9115 | 0.8983 |
| 3005 | PI | 0.4854 | 0.6466 | 0.5780 |
| 3005 | GI | 0.6516 | 0.7901 | 0.7255 |
| 3005 | BOP | 0.2796 | 0.2953 | 0.2868 |
| 3005 | PD | 0.9098 | 0.9111 | 0.9078 |

TABLE 3A
PERCENTAGE CHANGES IN MEASUREMENTS

| | | | | TOTAL |
|---|---|---|---|---|
| | | SCALED | UNSCALED | (6-point, 30 |
| | MEASURE- | (6-point, 15 | (6-point, 15 | tooth, |
| PATIENT | MENT | tooth, % diff.) | tooth, % diff.) | % diff.) |
| 3001 | PI | −12.4 | 4.6 | −4.1 |
| 3001 | GI | −53.0 | −53.5 | −53.3 |
| 3001 | BOP | −25.0 | −52.8 | −39.7 |
| 3001 | PD | 2.8 | −1.1 | 0.8 |
| 3002 | PI | −17.7 | 9.3 | −6.8 |
| 3002 | GI | −69.6 | −68.7 | −69.2 |
| 3002 | BOP | −71.0 | −80.2 | −75.8 |
| 3002 | PD | 0.5 | −1.5 | −0.5 |
| 3003 | PI | 0.0 | 7.7 | 3.2 |
| 3003 | GI | −48.5 | −62.1 | −55.5 |
| 3003 | BOP | −35.6 | −26.5 | −31.6 |
| 3003 | PD | −7.2 | −4.0 | −5.7 |
| 3004 | PI | −35.2 | 4.0 | −18.9 |
| 3004 | GI | −78.3 | −65.6 | −72.4 |
| 3004 | BOP | −42.9 | −57.1 | −50.0 |
| 3004 | PD | −11.3 | −4.5 | −8.0 |
| 3005 | PI | 138.5 | 147.4 | 143.8 |
| 3005 | GI | −76.5 | −68.9 | −72.4 |
| 3005 | BOP | −71.7 | −72.4 | −72.1 |
| 3005 | PD | 6.8 | −6.0 | 0.0 |
| MEAN | PI | 14.6 | 34.6 | 23.4 |
| MEAN | GI | −65.2 | −63.8 | −64.5 |
| MEAN | BOP | −49.2 | −57.8 | −53.9 |
| MEAN | PD | −1.7 | −3.4 | −2.7 |

TABLE 3B
P-VALUES FOR CHANGES IN MEASUREMENTS

| | | SCALED | UNSCALED | TOTAL |
|---|---|---|---|---|
| | MEASURE- | (6-point, 15 | (6-point, 15 | (6-point, 30 |
| PATIENT | MENT | tooth, pval) | tooth, pval) | tooth, pval) |
| 3001 | PI | 0.131243 | 0.617710 | 0.503626 |
| 3001 | GI | 0.000001 | 0.000000 | 0.000000 |
| 3001 | BOP | 0.192630 | 0.001490 | 0.001590 |
| 3001 | PD | 0.670348 | 0.865762 | 0.856690 |
| 3002 | PI | 0.182366 | 0.618267 | 0.537341 |
| 3002 | GI | 0.000000 | 0.000000 | 0.000000 |
| 3002 | BOP | 0.000042 | 0.000000 | 0.000000 |
| 3002 | PD | 0.917775 | 0.768154 | 0.886531 |
| 3003 | PI | 1.000000 | 0.781777 | 0.844476 |
| 3003 | GI | 0.000002 | 0.000000 | 0.000000 |
| 3003 | BOP | 0.010104 | 0.139089 | 0.004112 |
| 3003 | PD | 0.205367 | 0.490825 | 0.166052 |
| 3004 | PI | 0.000055 | 0.738630 | 0.008567 |
| 3004 | GI | 0.000000 | 0.000000 | 0.000000 |
| 3004 | BOP | 0.172104 | 0.057384 | 0.020939 |
| 3004 | PD | 0.019075 | 0.345231 | 0.018187 |
| 3005 | PI | 0.002038 | 0.000156 | 0.000001 |
| 3005 | GI | 0.000000 | 0.000000 | 0.000000 |
| 3005 | BOP | 0.000405 | 0.000077 | 0.0000000 |
| 3005 | PD | 0.320232 | 0.324766 | 1.0000000 |

TABLE 3C
CHANGES IN MEAN MEASUREMENTS FOR SCALED TEETH

| | | | SCALED (6- | PRE- | |
|---|---|---|---|---|---|
| | | SCALED (6- | point, | POST | PRE- |
| | | point, 15 | 15 tooth, | (% | POST (p |
| | MEASUREMENT | tooth, AVG) | STD) | diff.) | value) |
| PRE-TREAT. | PI | 0.9020 | 0.2111 | | |
| PRE-TREAT. | GI | 1.3641 | 0.2132 | | |
| PRE-TREAT. | BOP | 0.3639 | 0.2177 | | |
| PRE-TREAT. | PD | 2.8689 | 0.1791 | | |
| POST- | PI | 0.7804 | 0.4167 | 13.5 | 0.3284 |
| TREAT. | | | | | |
| POST- | GI | 0.4974 | 0.4725 | 63.5 | 0.000089 |
| TREAT. | | | | | |
| POST- | BOP | 0.1905 | 0.2254 | 47.7 | 0.0116 |
| TREAT. | | | | | |
| POST- | PD | 2.7772 | 0.2530 | 3.2 | 0.4092 |
| TREAT. | | | | | |

### Example 3

### Lack of Antibacterial Activity

Haemophilus actinomycetemcomitans (also known as Actinobacillus actinomycetemcomitans) and Porphyromonas gingivalis are anaerobic bacteria closely associated with the onset and severity of periodontal disease. Morinushi et al., J. Periodontal. 71(3):403-409 (March 2000).

Haemophilus actinomycetemcomitans (ATCC 29522) and Porphyromonas gingivalis (ATCC 33277) were obtained from the American Type Culture Collection (ATCC) and rehydrated and plated to the usual anaerobic media for growth. A 0.5 MacFarland standard suspension was prepared from a fresh subculture of each microorganism. A lawn of each microorganism suspension was plated to CDC Anaerobic Blood Agar and Chocolate Agar. The following concentrations of Syprine® trientine hydrochloride (Merck & Co., Inc.) were tested against each microorganism on each of the two media: 27.5 mg/L, 55 mg/L and 110 mg/L (the contents of one capsule of Syprine® trientine hydrochloride were dissolved in deionized water and filtered through a 0.45 micron filter to prepare these solutions). To do the testing, 0.25 ml of each of the three concentrations of Syprine® trientine hydrochloride was added to a sterile disk on each plate. The plates were incubated under anaerobic conditions (Gas Pak Pouch system) for a total of 48 hours. No zone of inhibition was present on any of the plates. Thus, Syprine® trientine hydrochloride at these concentrations did not inhibit the growth of these two microorganisms. The mouth rinse used in Example 2 contained 55 mg/L of trientine hydrochloride.

1. A method of inhibiting the release of pro-inflammatory cytokines from cells located in a tissue of an animal's mouth comprising contacting the tissue with an effective amount of a non-peptide polyamine chelating agent or a physiologically-acceptable salt thereof.

2-13. (Canceled)

14. A method of treating inflammation of a tissue of an animal's mouth comprising contacting the tissue with an effective amount of a non-peptide polyamine chelating agent or a physiologically-acceptable salt thereof.

15-17. (Canceled)

18. A method of treating an inflammatory disease or condition of a tissue of an animal's mouth comprising contacting the tissue with an effective amount of a non-peptide polyamine chelating agent or a physiologically-acceptable salt thereof.

19. The method of claim 18 wherein the disease or condition is gingivitis.

20. The method of claim 18 wherein the disease or condition is periodontitis.

21. The method of claim 18 wherein the tissue is contacted with the non-peptide polyamine chelating agent or physiologically-acceptable salt thereof as part of a prophylactic oral regimen.

22. The method of claim 18 wherein the non-peptide polyamine chelating agent or physiologically-acceptable salt thereof is contained in an oral care composition.

23. The method of claim 22 wherein the oral care composition is a mouthwash or mouth rinse.

24. The method of claim 22 wherein the oral care composition is a gargle, a spray or a solution.

25. The method of claim 22 wherein the oral care composition is a gel, a paste or a powder.

26. The method of claim 22 wherein the oral care composition is an ointment or a cream.

27. The method of claim 22 wherein the oral care composition is a gum, a lozenge, or a mint.

28. The method of claim 22 wherein the oral care composition is a tooth whitening composition.

29. The method of claim 18 wherein the non-peptide polyamine chelating agent or physiologically-acceptable salt thereof is contained in an oral care device.

30. A method of reducing the damage done by reactive oxygen species (ROS) to a tissue of an animal's mouth comprising contacting the tissue with an effective amount of a non-peptide polyamine chelating agent or a physiologically-acceptable salt thereof.

31-35. (Canceled)

36. A method of whitening one or more teeth of an animal comprising contacting the one or more teeth, another tissue of the animal's mouth or both with an effective amount of a non-peptide polyamine chelating agent or a physiologically-acceptable salt thereof.

37-42. (Canceled)

43. The method of any one of claims 1, 14, 18-30 or 36 wherein the non-peptide polyamine chelating agent has the following formula:
NR¹R²[—(CR³R⁴)ₓ—NR¹]_{y}—(CR³R⁴)ₓ—NR¹R²
wherein:
each x may independently be 2 or 3;
y is 1-10;
each R¹, R², R³ and R⁴ may be the same or different;
each R¹, R², R³ and R⁴ may be H, alkyl, alkenyl, aryl, arylalkyl, alkoxy, alkenoxy, aryloxy, alkyl interrupted by one or more oxygen atoms (—O—), alkyl interrupted by one or more nitrogen atoms (—N—), alkenyl interrupted by one or more oxygen atoms (—O—), alkenyl interrupted by one or more nitrogen atoms (—N—), aryloxyalkyl, alkoxyaryl, aminoalkyl, aminoalkenyl, aminoaryl, aminoarylalkyl, hydroxyalkyl, hydroxyalkenyl, hydroxyaryl or hydroxyarylalkyl; and
each R¹ and R² may also be:
—CR⁵R⁶—(CR⁷R⁸)_{z}—R⁹
wherein:
each R⁵, R⁶ and R⁷ may be the same or different, and each R⁵, R⁶ or R⁷ may be H, alkyl, alkenyl, aryl, arylalkyl, alkoxy, alkenoxy, aryloxy, alkyl interrupted by one or more oxygen atoms (—O—), alkyl interrupted by one or more nitrogen atoms (—N—), alkenyl interrupted by one or more oxygen atoms (—O—), alkenyl interrupted by one or more nitrogen atoms (—N—), aryloxyalkyl, alkoxyaryl, aminoalkyl, aminoalkenyl, aminoaryl, aminoarylalkyl, hydroxyalkyl, hydroxyalkenyl, hydroxyaryl or hydroxyarylalkyl;
R⁸ may be H, hydroxy, amino, alkyl, alkyl interrupted by one or more oxygen atoms (—O—), alkoxy, aryl, aryloxyalkyl or alkoxyaryl;
R⁹ may be —COOR¹⁰, —CON(R¹⁰)₂, —OR¹⁰, —C(OR¹⁰)₃, —COR¹⁰, —N(R¹⁰)₂, alkyaryl, or alkylheteroaryl;
R¹⁰ may be H, alkyl, aryl or heteroaryl; and
z is 0-6.

44. The method of claim 43 wherein R¹, R², R³ and R⁴ are all H, each x is independently 2 or 3, and y is 1-5.

45. The method of claim 44 wherein the non-peptide polyamine chelating agent is diethylenetriamine, trientine, tetraethylenepentamine, pentaethylenehexamine, hexaethyleneseptamine, dipropylenetriamine, tripropylenetetramine, tetrapropylenepentamine, pentapropylenehexamine, hexapropyleneseptamine, N,N′-bis(2-aminoethyl)propanediamine orN,N′-bis(2-aminopropyl)ethanediamine.

46. The method of claim 45 wherein the non-peptide polyamine chelating agent is trientine.

47. A method of treating a tissue of an animal's mouth comprising contacting the tissue with from about 0.002 mg to about 2.20 mg of trientine or a physiologically-acceptable salt thereof.

48-51. (Canceled)

52. A method of treating a disease or condition of a tissue of an animal's mouth comprising contacting the tissue with from about 0.002 mg to about 2.20 mg of trientine or a physiologically-acceptable salt thereof.

53-58. (Canceled)

59. The method of any one of claims 1, 14, 18, 30, 36, 47 or 52 wherein the animal is a human.

60. The method of any one of claims 1, 14, 18, 30, 36, 47 or 52 wherein the method further comprises contacting the tissue with an effective amount of another metal-binding compound.

61. An oral care device comprising a non-peptide polyamine chelating agent or a physiologically-acceptable salt thereof.

62-69. (Canceled)

70. An oral care composition comprising a pharmaceutically-acceptable carrier and from about 0.001% to about 25% by weight of a non-peptide polyamine chelating agent or a physiologically-acceptable salt thereof.

71. The composition of claim 70 wherein the non-peptide polyamine chelating agent has the following formula:
NR¹R²[—(CR³R⁴)ₓ—NR¹]_{y}—(CR³R⁴)ₓ—NR¹R²
wherein:
each x may independently be 2 or 3;
y is 1-10;
each R¹, R², R³ and R⁴ may be the same or different;
each R¹, R², R³ and R⁴ may be H, alkyl, alkenyl, aryl, arylalkyl, alkoxy, alkenoxy, aryloxy, alkyl interrupted by one or more oxygen atoms (—O—), alkyl interrupted by one or more nitrogen atoms (—N—), alkenyl interrupted by one or more oxygen atoms (—O—), alkenyl interrupted by one or more nitrogen atoms (—N—), aryloxyalkyl, alkoxyaryl, aminoalkyl, aminoalkenyl, aminoaryl, aminoarylalkyl, hydroxyalkyl, hydroxyalkenyl, hydroxyaryl or hydroxyarylalkyl; and
each R¹ and R² may also be:
—CR⁵R⁶—(CR⁷R⁸)_{z}—R⁹
wherein:
each R⁵, R⁶ and R⁷ may be the same or different, and each R⁵, R⁶ or R⁷ may be H, alkyl, alkenyl, aryl, arylalkyl, alkoxy, alkenoxy, aryloxy, alkyl interrupted by one or more oxygen atoms (—O—), alkyl interrupted by one or more nitrogen atoms (—N—), alkenyl interrupted by one or more oxygen atoms (—O—), alkenyl interrupted by one or more nitrogen atoms (—N—), aryloxyalkyl, alkoxyaryl, aminoalkyl, aminoalkenyl, aminoaryl, aminoarylalkyl, hydroxyalkyl, hydroxyalkenyl, hydroxyaryl or hydroxyarylalkyl;
R⁸ may be H, hydroxy, amino, alkyl, alkyl interrupted by one or more oxygen atoms (—O—), alkoxy, aryl, aryloxyalkyl, or alkoxyaryl;
R⁹ may be —COOR¹⁰, —CON(R¹⁰)₂, —OR¹⁰, —C(OR¹⁰)₃, —COR¹⁰, —N(R¹⁰)₂, alkyaryl, or alkylheteroaryl;
R¹⁰ may be H, alkyl, aryl or heteroaryl; and
z is 0-6.

72. The composition of claim 71 wherein R¹, R², R³ and R⁴ are all H, each x is independently 2 or 3, and y is 1-5.

73. The composition of claim 72 wherein the non-peptide polyamine chelating agent is diethylenetriamine, trientine, tetraethylenepentamine, pentaethylenehexamine, hexaethyleneseptamine, dipropylenetriamine, tripropylenetetramine, tetrapropylenepentamine, pentapropylenehexamine, hexapropyleneseptamine, N,N′-bis(2-aminoethyl)propanediamine orN,N′-bis(2-aminopropyl)ethanediamine.

74. The composition of claim 73 wherein the non-peptide polyamine chelating agent is trientine.

75. The composition of any one of claims 70-74 comprising from about 2.5% to about 12.5% of the non-peptide polyamine chelating agent or physiologically-acceptable salt thereof.

76. The composition of claim 75 comprising from about 5.0% to about 6.0% of the non-peptide polyamine chelating agent or physiologically-acceptable salt thereof.

77. The composition of any one of claims 70-76 wherein the composition is a wash, a rinse, a gargle, a spray or a solution.

78. The composition of any one of claims 70-76 wherein the composition is a gel, a paste or a powder.

79. The composition of any one of claims 70-76 wherein the composition is an ointment or a cream.

80. The composition of any one of claims 70-76 wherein the composition is a gum, a lozenge or a mint.

81. A tooth whitening composition comprising a pharmaceutically-acceptable carrier and a non-peptide polyamine chelating agent or a physiologically-acceptable salt thereof.

82-86. (Canceled)

87. A kit comprising an oral care product, the oral care product comprising a non-peptide polyamine chelating agent or a physiologically-acceptable salt thereof.

88-101. (Canceled)
